(19) United States
(12) Patent Application Publication
(10) Pub. No.: US 2023/0292750 A1
(43) Pub. Date: Sep. 21, 2023

(54) INSECT REPELLENT PATCHES AND TUBULAR ARTICLES

(71) Applicant: ACHIEVE EQUINE LLC, Delano, MN (US)

(72) Inventors: James R. Chiapetta, Delano, MN (US); Edward Parsonage, St. Paul, MN (US)

(21) Appl. No.: 18/017,204

(22) PCT Filed: Aug. 3, 2021
(86) PCT No.: PCT/US2021/044396

§ 371 Date: Jan. 20, 2023

Related U.S. Application Data
(60) Provisional application No. 63/060,945, filed on Aug. 4, 2020.

(51) Int. Cl. A01N 25/34; A01N 25/18; A01N 25/10

(52) U.S. Cl. CPC A01N 25/34; A01N 25/18; A01N 25/10

(57) ABSTRACT

An insect repellent articles, such as tubular articles and patches, wherein an exemplary patch includes: a support film having a first major surface and a second major surface; a body-contacting pressure sensitive adhesive layer disposed on the first major surface; and a reservoir layer disposed on the second major surface, wherein the reservoir layer comprises an insect repellent incorporated within a polymeric carrier, and wherein the reservoir layer forms a non-contacting surface.

### BACKGROUND

Various articles designed to release vapors or liquids from a compound or combinations of compounds have been in wide use over the years. For example, perfume patches or pads provide a temporary short term pleasant odor to the environment in the immediate vicinity of the patch or pad. The samples typically consist of the perfume absorbed on a paper-type support. The perfumes have a rapid rate of release during the initial phase of their life and a significantly reduced rate of release in the later stages.

Controlled release patches are used extensively in the pharmaceutical industry to provide therapeutic and diagnostic compounds to patients over periods of time ranging from minutes to days. In particular, skin patches have been used successfully in administering medications transdermally for several hours at a time. For example, a patch for delivering the sea sickness medication, scopolamine, is a five layer laminate designed to be attached to the skin and deliver the drug through the patch's adhesive layer and into the user's circulatory system.

In addition to these, other articles have been used to release insect repellents on animals for decades. For example, flea and tick repellent and insecticides are incorporated in pet collars and then released onto the pet while the pet is wearing the collar to help control fleas and ticks that come into contact with the animal. Such collars are particularly adaptable for pets because it is difficult to apply insect repellents and insecticides to animal skin and fur on a regular basis.

Additionally, people have used insect repellents directly on the skin for over 30 years. Moreover, the use of insect repellents has increased dramatically in recent years in conjunction with the heightened concern associated with Lyme disease. Known topical methods for delivering insect repellents in a manner that is safe and efficacious may be dangerous, short-acting, and inconvenient. Another problem associated with topically applying insect repellents directly to the skin is that they are effective for only short periods of time. Because the insect repellent is removed by skin absorption, evaporation, perspiration, and through participation in water activities, it must be reapplied at frequent intervals.

There is still a need for articles that will release insect repellent into the environment surrounding the user.

### SUMMARY OF THE DISCLOSURE

The present disclosure provides insect repellent articles, particularly patches and tubular articles.

Generally, an insect repellent patch includes: a support film having a first major surface and a second major surface; a body-contacting pressure sensitive adhesive layer disposed on the first major surface; and a reservoir layer disposed on the second major surface, wherein the reservoir layer includes an insect repellent incorporated within a polymeric carrier, and wherein the reservoir layer forms a non-contacting surface.

In one embodiment, an insect repellent patch is provided that includes: an occlusive support film having a first major surface and a second major surface; a body-contacting pressure sensitive adhesive layer disposed on the first major surface; and a reservoir layer disposed on the second major surface, wherein the reservoir layer includes an insect repellent incorporated within a nonporous diffusion controlling polymeric carrier; wherein the polymeric carrier includes polyisobutene, polybutadiene, polystyrene-isoprene copolymer, polystyrene-butadiene (SBR) copolymer, polychloroprene (neoprene), isoprene (i.e., 2-methyl-1,3-butadiene), or a combination thereof; and wherein the reservoir layer forms a non-contacting surface.

In one embodiment, an insect repellent patch is provided that includes: a support film having a first major surface and a second major surface; a body-contacting pressure sensitive adhesive layer disposed on the first major surface; and a reservoir layer disposed on the second major surface, wherein the reservoir layer includes an insect repellent incorporated within a polymeric carrier, and wherein the reservoir layer forms a non-contacting surface; and further wherein the patch is configured of a size and shape for a horse's face. In one embodiment, the patch is a pentagonal configuration, which, for example, is shaped to follow contours of a mid to caudal region of a horse's face. In another embodiment, the patch is a trapezoidal configuration, which, for example, is shaped to follow contours of a poll region of a horse's face and to repel insects particularly from the eyes and ears of the animal.

In another embodiment is an insect repellent tubular article that includes: a tubular structure having a first major surface and a second major surface, wherein the tubular structure comprises an elastic or shape memory material; wherein the first major surface provides a reservoir comprising an insect repellent, and the second major surface provides a body-contacting surface. In certain embodiments, the body-contacting surface of the tubular structure is coated with a barrier.

As used herein, the terms “polymer” and “polymeric” and “polymeric material” are used interchangeably to refer to a homopolymer, copolymer, terpolymer, and the like.

As used herein, the term “(meth)acrylate” refers to both methacrylate and acrylate monomers, polymeric materials derived from these monomers, or both. Likewise, the term “(meth)acrylic” refers to both acrylic and methacrylic materials.

The term “glass transition temperature” or “Tg” refers to the temperature at which a material changes from a glassy state to a rubbery state. In this context, the term “glassy” means that the material is hard and brittle (and therefore relatively easy to break) while the term “rubbery” means that the material is elastic and flexible. For polymeric materials, the Tg is the critical temperature that separates their glassy and rubbery behaviors. If a polymeric material is at a temperature below its Tg, large-scale molecular motion is severely restricted because the material is essentially frozen. On the other hand, if the polymeric material is at a temperature above its Tg, molecular motion on the scale of its repeat unit takes place, allowing it to be soft or rubbery. The glass transition temperature of a polymeric material is often determined using methods such as Differential Scanning calorimetry (e.g., Modulated Differential Scanning calorimetry). Alternatively, the glass transition of a polymeric material can be a Fox Tg. The Fox Tg is an estimated (i.e., calculated) value using the Fox equation. For example, for a polymer made from two monomers, the theoretical Tg may be calculated using the Fox equation as follows:

1/Tg=Wa/Tga+Wb/Tgb

wherein:

Tga and Tgb are the respective glass transition temperatures in Kelvin of homopolymers made from monomers “a” and “b”; and

Wa and Wb are the respective weight fractions of polymers “a” and “b”. When additional monomers “c” and “d” and so on are employed, additional fractions Wc/Tgc, Wd/Tgd and so on are added to the right-hand side of the above equation.

The value of Tg of the monomers used to estimate the polymer Tg are based on literature values. Typically, there is some variation of the Tg values of the homopolymers of monomers listed in such literature. The difference arises from the test method used to measure the Tg. The differences also arise from influence of comonomers polymerized together. Alternatively, the method of determining the Tg of a homopolymer can be determined using a DSC procedure.

Herein, the term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other claims are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

As used herein, the term “room temperature” refers to a temperature of 20° C. to 25° C.

The term “in the range” or “within a range” (and similar statements) includes the endpoints of the stated range.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF FIGURES

FIG. 1 is a cross-sectional representations of an exemplary patch.

FIG. 2 is a front view of a horse's head illustrating configurations of repellent patches according to the disclosure.

FIG. 3 is a front view of a horse's head illustrating an alternative configuration of a repellent patch according to the disclosure.

FIG. 4 is a ventral view of a horse's body illustrating configurations of repellent patches according to the disclosure.

FIG. 5 is a rear view of a horse's body illustrating configurations of repellent patches according to the disclosure.

FIG. 6 is a front view of a horse's body illustrating configurations of repellent patches according to the disclosure.

FIG. 7 is a top view of a horse's body illustrating a configuration of a repellent patch according to the disclosure.

FIG. 8 is a side view of a horse's body illustrating tubular repellent articles on the horse's front and rear leg's according to the disclosure.

FIG. 9 is a cross-sectional view through line 9-9 of FIG. 8 illustrating ribs inside a tubular repellent article according to the disclosure.

### DETAILED DESCRIPTION OF ILLUSTRATIVE EMBODIMENTS

The present disclosure provides insect repellent articles.

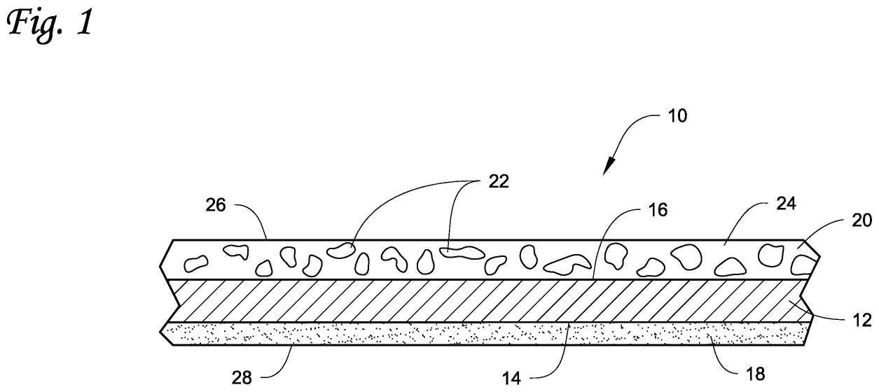

Referring to FIG. 1, an insect repellent patch 10 is shown that includes: a support film 12 having a first major surface 14 and a second major surface 16; a pressure sensitive adhesive layer 18 disposed on the first major surface 14; and a reservoir layer 20 disposed on the second major surface 16. The reservoir layer 20 includes an insect repellent 22 incorporated within a polymeric carrier 24. The reservoir layer 20 forms a non-contacting surface 26 and the adhesive layer 18 forms a body-contacting surface 28 (i.e., a surface that contacts the body of the wearer). Optionally, disposed on the reservoir layer's non-contacting surface 26 may be a removable reservoir liner (not shown) and disposed on the adhesive layer's body-contacting surface 28 may be an adhesive liner (not shown).

The insect repellent articles of the present disclosure can be used on a variety of subjects (referred to herein as the wearer or user), whether they be human or animal. Exemplary animals include, horses, cows, pigs, dogs, cats, camels and other domesticated or non-domesticated animals. In embodiments, the insect repellent patch may be particularly beneficial for horses and cattle.

During use, the insect repellent article dispenses the insect repellent in close proximity to the wearer. That is, the insect repellent is released to the surroundings of the wearer (e.g., in a plume around the head of a horse).

In the context of a patch (e.g., as shown in FIG. 1), a “non-contacting surface” is a surface that does not contact the body of the wearer (i.e., user). Thus, the reservoir layer does not contact the body of the wearer and the insect repellent is not administered into the wearer. That is, the patch of the present disclosure is not a transdermal patch that administers an active agent through the patch's adhesive layer and into the user's circulatory system.

In certain embodiments of a patch (e.g., as shown in FIG. 1), the polymeric carrier, insect repellent, and support film are selected to pass the Adhesion Test. To pass this test, the polymeric carrier must adhere to the support film to maintain structural integrity of the patch. The degree of desired adhesion can be defined as cohesive failure of the interface on separation, with transfer of the polymeric carrier to the support film during peel. For example, this may be tested by trying to separate the carrier substrate interface with a sharpened probe as demonstrated in the Adhesion Test.

In certain embodiments of a patch (e.g., as shown in FIG. 1), the polymeric carrier, insect repellent, and support film are selected to pass the Tack Test. Although the reservoir layer is preferably tacky, it should not be too tacky. If the reservoir layer is too tacky, the patch could, for example, stick to a tree and pull off an animal or adhere to environmental contaminants (e.g., sand, dust, dirt, debris, and hair). The level of tack can be defined as interfacial adhesive failure when the surface of the polymeric carrier of the repellent layer is probed with a biomaterial (e.g., wooden dowel) as shown in the Tack Test. Per this test, if probed in a controlled manner with a wooden dowel, the interface between the dowel and the surface of the polymeric carrier of the reservoir layer does not cohesively fail with transfer of the carrier material to the dowel.

In certain embodiments, an article, particularly a patch, of the present disclosure is effective for repelling insects for a period of at least 1 day, at least 2 days, at least 7 days, at least 14 days, or at least 1 month. In certain embodiments, an article, particularly a patch, of the present disclosure is effective for repelling insects for a period of up to 3 months, up to 21 days, up to 14 days, or up to 7 days.

Reservoir Layer

In the context of a patch (e.g., as shown in FIG. 1), a reservoir layer includes an insect repellent incorporated within a polymeric carrier, wherein the reservoir layer forms a non-contacting surface. In this context, a “non-contacting surface” is a surface that does not contact the body of the wearer (i.e., user).

Preferably, the polymeric carrier is a nonporous diffusion controlling polymeric carrier. In this context, “nonporous” means that there are no spaces or holes in the polymeric carrier (as in a fibrous, e.g., woven or nonwoven, material) through which liquid, vapor, or air may pass, although the polymeric carrier is permeable to the insect repellent. Preferably, the reservoir layer does not include a fibrous material or a foam pad.

A “diffusion controlling” polymeric carrier is one that inherently controls diffusion of the insect repellent without the need for a rate-limiting membrane disposed on the reservoir layer.

The polymeric carrier is selected to allow transmission of the insect repellent (e.g., in liquid or vapor form) out of the reservoir layer and released to the surroundings of, and in close proximity to, the wearer. For example, the polymeric carrier is selected such that the insect repellent is released in a plume around the head of a horse.

Thus, the polymeric carrier of the reservoir layer is selected to solubilize the insect repellent and be permeable to the insect repellent and release it to the surroundings of the wearer. The release of the repellent is rate limited by diffusion through the polymeric carrier. The permeability of the repellent within the carrier material should be such as to allow an efficacious amount of release over a desired duration of time. The release rate required for a repellent to be efficacious will depend on the specific chemical agent.

In certain embodiments, the components of the reservoir layer are selected to allow the insect repellent to release from the reservoir layer over a period of at least 1 day, at least 2 days, at least 7 days, at least 14 days, or at least 1 month. In certain embodiments, the components of the reservoir layer are selected to allow the insect repellent to release from the reservoir layer over a period of up to 3 months, up to 21 days, up to 14 days, or up to 7 days.

Furthermore, the polymeric carrier of the reservoir layer is selected to adhere to the support layer but not adhere to environmental contaminants (e.g., sand, dust, dirt, debris, and hair) due to too tacky of a surface (according to the Tack Test).

In certain embodiments, the polymeric carrier of the reservoir layer includes a rubber-based polymer, an unsaturated polyolefin (such as polyisobutene, polybutadiene, cis- and trans-polyisoprene, and chloroprene elastomers), a copolymer of unsaturated polyolefins (such as styrene-butadiene copolymer, styrene-isoprene copolymer, and acrylonitrile-butadiene-styrene copolymers), a vinyl polymer (such as polyvinyl chloride), a (meth)acrylate (such as poly-n-butyl acrylate), or a combination thereof.

In certain embodiments, the polymeric carrier does not include an ethylene-containing polymer (such as ethylene-vinyl acetate copolymers).

In certain embodiments, the polymeric carrier of the reservoir layer includes a rubber-based polymer, an unsaturated polyolefin (such as polyisobutene, polybutadiene, cis- and trans-polyisoprene, and chloroprene elastomers), a copolymer of unsaturated polyolefins (such as styrene-butadiene copolymer, styrene-isoprene copolymer, and acrylonitrile-butadiene-styrene copolymers), or a combination thereof.

In certain embodiments, the carrier polymer includes polyisobutene, polybutadiene, polystyrene-isoprene copolymer, polystyrene-butadiene (SBR) copolymer, polychloroprene (neoprene), isoprene (i.e., 2-methyl-1,3-butadiene), or a combination thereof.

In certain embodiments, the carrier polymer includes a polystyrene-butadiene (SBR) copolymer. A preferred SBR is a low surface tack SBR available as CAMI 390B from Camie-Campbell Inc.

In certain embodiments, the polymeric carrier of the reservoir layer is crosslinked. This may result from chemical crosslinking (e.g., through chain transfer to polymer chains (or polymer chains losing labile tertiary hydrogens) and/or physical crosslinking (e.g., ionic crosslinking) of the polymer, or it may result from the addition of a separate (secondary) crosslinker (i.e., crosslinking agent) such as a thermal crosslinker that reacts with functional groups of the polymer.

In certain embodiments, the reservoir layer may further include one or more optional additives. Such additives may be selected from a tackifier, plasticizer, UV absorber, antioxidant, sunscreen, synergist, stabilizer, or a combination thereof.

In certain embodiments, the reservoir layer further includes a synergist. Exemplary synergists include piperonyl butoxide, di-n-propyl isocynchomeronate, buoxypolypropylene glycol, bicycloheptene dicaroxamide, and a combination thereof.

In certain embodiments, the reservoir layer further includes a tackifier. Exemplary tackifiers include a rosin or derivative thereof, a terpene or modified terpene, an aliphatic resin (e.g., C5-aliphatic resin), a cycloaliphatic resin (e.g., C6-cycloaliphatic resin), an aromatic resin (e.g., C9-aromatic resin), a hydrogenated hydrocarbon resin, a novolac resin, and a combination thereof (e.g., C5/C9 aliphatic/aromatic resin, terpene-phenol (TPR) resin).

In certain embodiments, the reservoir layer further includes a plasticizer. Exemplary plasticizers include a phthalate, a trimellitate, an adipate, and a combination thereof.

In certain embodiments, the reservoir layer further includes a stabilizer. Exemplary stabilizers include a light stabilizer (e.g., oxybenzone), antioxidant (e.g., hindered amine and phenol), and a combination thereof.

The amount of each individual optional additive can be readily determined by one of skill in the art. In certain embodiments, the total amount of one or more optional additives present is at least 1 wt-%, and even up to 75 wt-%, based on the total weight of the components of the reservoir layer.

Typically, the reservoir layer is coextensive with the support film (i.e., the reservoir layer is disposed on the entirety of the support film).

In certain embodiments, the reservoir layer has a uniform thickness throughout its length and width. In certain embodiments, the reservoir layer has a thickness of at least 0.005 inch (0.013 mm). In certain embodiments, the reservoir layer has a thickness of up to 0.025 inch (0.64 mm).

Insect Repellent

In the articles, e.g., patches, of the present disclosure, the insect repellent includes one or more compounds (i.e., chemical agents) that effectively repel insects. In certain embodiments, the insect repellent includes a naturally occurring compound, a synthetic compound, or a combination thereof.

Preferably, the insect repellent has a vapor pressure sufficient to allow the insect repellent to diffuse out of the article (e.g., reservoir layer of a patch) and form a plume of repellent that “sits around” the animal's head (some bugs need to touch it and then fly away). A typical vapor pressure is 10-8 mm Hg to 10-3 mm Hg.

In certain embodiments, the insect repellent includes a naturally occurring pyrethrum extract, a synthetic pyrethroid, a naturally occurring oil, a terpene, a terpenoid, an alkanoic acid, an organophosphate, a toluamide, or a combination thereof. In certain embodiments, the insect repellent includes a naturally occurring pyrethrum extract, a synthetic pyrethroid, or a combination thereof. In certain embodiments, the insect repellent includes permethrin, Cypermethrin, metofluthrin, profluthrin, transfluthrin, or a combination thereof.

In certain embodiments, the insect repellent includes a naturally occurring oil. In certain embodiments, the naturally occurring oil includes lemon eucalyptus oil, clove oil, cottonseed oil, neem oil, lemongrass oil, citronella oil, cedarwood oil, lavender oil, rosemary oil, coconut oil, fir needle oil, litsea cubeba oil, pyganic (chrysanthemum) oil, or a combination thereof.

In certain embodiments, the insect repellent includes a terpene (e.g., pulegone), a terpenoid (e.g., geraniol), an alkanoic acid (e.g., a C8-C10-alkanoic acid), an organophosphate (e.g., 2,2-dichlorovinyl dimethyl phosphate), a toluamide (e.g., N,N-diethyl-meta-toluamide (i.e., DEET)), or a combination thereof.

In certain embodiments, the insect repellent is present in the polymeric carrier of the reservoir layer of a patch in an amount of at least 1 wt-%, or at least 5 wt-%, or at least 10 wt-%, based on the total weight of the reservoir.

In certain embodiments, the insect repellent is present in the polymeric carrier of the reservoir layer of a patch in an amount of up to 90 wt-%, or up to 85 wt-%, or up to 80 wt-%, or up to 75 wt-%, or up to 70 wt-%, or up to 65 wt-%, based on the total weight of the reservoir.

Support Film

In the context of a patch (e.g., as shown in FIG. 1), a support film is selected to be sufficiently flexible to conform to a wearer's (i.e., user's) body contours, and such that it can be worn comfortably with little or no likelihood of the patch disengaging from the wearer due to differences in flexibility or resiliency. It is often made of a polymeric film, fabric, foam, paper, or metal-containing film.

Preferably, the support film is occlusive. An occlusive film allows little or no transmission of the insect repellent (e.g., in liquid or vapor form) from the reservoir layer into the adhesive layer (e.g., such that the insect repellent does not negatively affect the adhesive) and through the adhesive layer to contact the skin and/or hair of the wearer. That is, the patch dispenses the insect repellent in close proximity to the wearer while not bringing the insect repellent into contact with the wearer by migrating through the support film and adhesive layer. For example, the insect repellent may be released to the surroundings of the wearer (e.g., in a plume around the head of a horse). That is, the support film possesses little or no permeability with respect to the insect repellent.

In certain embodiments, the support film is made of an organic polymer (e.g., thermoplastic resin or thermoset resin), a metal-containing material (e.g., metal such as A1 or other thin metal film, metal alloy, vapor-deposited metal layer, or shape memory metal), or a combination thereof.

Typically, organic polymers, particularly thermoplastic resins, that can form occlusive films include materials having relatively high glass transition temperatures (e.g., Tg of at least 30° C.), relatively high crystallinity (e.g., at least 15%), and low repellent solubility (i.e., the repellent has little or no solubility in the polymer).

In certain embodiments, the thermoplastic resin is selected from poly(meth)acrylates (such as the polymethylmethacrylate, poly-n-butyl acrylate, poly(ethylene-co-acrylic acid), poly(ethylene-co-methacrylate), etc.), fluoropolymers (such as polytetrafluoroethylene (PTFE) (e.g., that available under the tradename TEFLON), poly(ethylene-co-tetrafluoroethylene) (ETFE) copolymers, (tetrafluoroethylene-co-propylene) (FEP) copolymers, polyvinylidine fluoride (PVDF) polymers, etc.), perfluoroalkoxy alkane (PFA) polymers, polyamides (such as nylon-6, nylon-6,6, etc.), polycarbonates, polyesters (such as poly(ethylene-co-terephthalate), poly(ethylene-co-1,4-naphthalene dicarboxylate), polysulfones, polyketones, polyurethanes, polyimide materials, poly(acrylonitrile-co-butadiene-co-styrene) polymers, poly(butylene-co-terephthalate), polyethylene materials (such as low density polyethylene, linear low density polyethylene, high density polyethylene, high molecular weight high density polyethylene, etc.), polypropylene (such as biaxially oriented polypropylene), polystyrene (such as biaxially oriented polystyrene), vinyl resins (such as polyvinyl chloride, (vinyl chloride-co-vinyl acetate) copolymers, polyvinylidene chloride, polyvinyl alcohol, (vinyl chloride-co-vinylidene dichloride) copolymers), polyphenylene sulfides, polyphenylene oxides, liquid crystal polyesters, polyether ketones, polyvinylbutyral, and combinations thereof.

In certain embodiments, the thermoplastic resin is selected from poly(meth)acrylates (such as the polymethylmethacrylate, poly-n-butyl acrylate, poly(ethylene-co-acrylic acid), poly(ethylene-co-methacrylate), etc.), fluoropolymers (such as polytetrafluoroethylene (PTFE) (e.g., that available under the tradename TEFLON), poly(ethylene-co-tetrafluoroethylene) (ETFE) copolymers, (tetrafluoroethylene-co-propylene) (FEP) copolymers, polyvinylidine fluoride (PVDF) polymers, etc.), perfluoroalkoxy alkane (PFA) polymers, polyamides (such as nylon-6, nylon-6,6, etc.), polycarbonates, polyesters (such as poly(ethylene-co-terephthalate), poly(ethylene-co-1,4-naphthalene dicarboxylate), polysulfones, polyketones, polyurethanes, polyimide materials, and combinations thereof.

In certain embodiments, the support film has a uniform thickness throughout its length and width. The thickness is typically selected based on the support needed. In certain embodiments, the support film has a thickness of at least 0.008 inch (0.20 mm). In certain embodiments, the support film has a thickness of up to 0.020 inch (0.51 mm).

The pressure sensitive and the reservoir layer are disposed on (i.e., positioned adjacent to and adhered to) opposite sides of the support film. The pressure sensitive adhesive layer may be directly or indirectly disposed on the first major surface of the support film. The reservoir layer may be directly or indirectly disposed on the second major surface of the support film.

In certain embodiments, the pressure sensitive adhesive layer is directly disposed on the first major surface of the support film. In certain embodiments, there is a primer layer therebetween. In certain embodiments, the reservoir layer is directly disposed on the second major surface of the support film. In certain embodiments, there is a primer layer therebetween.

The reservoir layer may be directly or indirectly disposed on the second major surface of the support film. In certain embodiments, either or both of the first major surface and second major surface of the support film is a treated surface to enhance adhesion of the layer(s) disposed thereon (e.g., the adhesive layer and the reservoir layer). Examples of treated surfaces are those that are corona treated, plasma treated, or chemically treated.

Pressure Sensitive Adhesive Layer

In the context of a patch (e.g., as shown in FIG. 1), a pressure sensitive adhesive is a “body-contacting adhesive.” By this, it is meant that the adhesive adheres to the body of a subject (i.e., the wearer) whether it be the skin or hair or both.

A variety of methods can be used to identify a pressure sensitive adhesive that may be used to identify a suitable pressure sensitive adhesive for use in the patches of the present disclosure.

One well known means of identifying pressure sensitive adhesives is the Dahlquist criterion. This criterion defines a pressure sensitive adhesive as an adhesive having a 1 second creep compliance of greater than 1×10⁻⁶ cm²/dyne as described in Handbook of Pressure Sensitive Adhesive Technology, Donatas Satas (Ed.), 2^{nd} Edition, p. 172, Van Nostrand Reinhold, New York, NY, 1989. Alternatively, since modulus is, to a first approximation, the inverse of creep compliance, pressure sensitive adhesives may be defined as adhesives having a Young's modulus of less than 1×10⁶ dynes/cm².

Another well known means of identifying a pressure sensitive adhesive is that it is aggressively and permanently tacky at room temperature and firmly adheres to a variety of dissimilar surfaces upon mere contact without the need of more than finger or hand pressure, and which may be removed from smooth surfaces without leaving a residue as described in Glossary of Terms Used in the Pressure Sensitive Tape Industry provided by the Pressure Sensitive Tape Council, August, 1985.

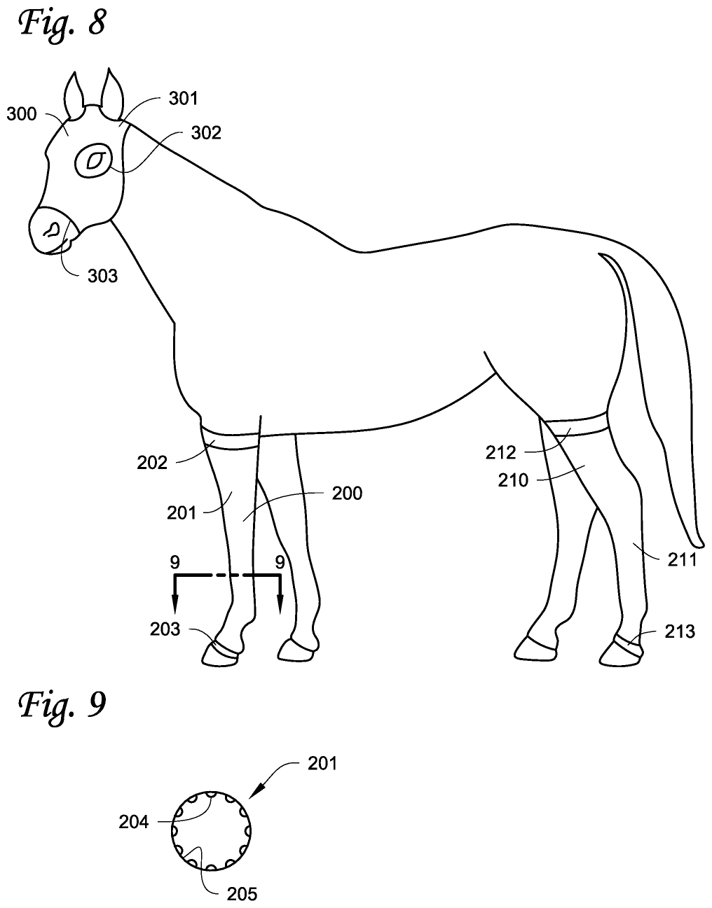

Another suitable definition of a suitable pressure sensitive adhesive is that it preferably has a room temperature storage modulus within the area defined by the following points as plotted on a graph of modulus versus frequency at 25° C.: a range of moduli from approximately 2×10⁵ to 4×10⁵ dynes/cm² at a frequency of approximately 0.1 radian/second (0.017 Hz), and a range of moduli from approximately 2×10⁶ to 8×10⁶ dynes/cm² at a frequency of approximately 100 radians/second (17 Hz) (for example, see FIG. 8-16 on p. 173 of Handbook of Pressure Sensitive Adhesive Technology (Donatas Satas, Ed.), 2nd Edition, Van Nostrand Rheinhold, New York, 1989).

Preferably, the pressure sensitive adhesive is selected to adhere to an animal's body (particularly, a horse) and the skin and/or hair thereon.

In certain embodiments, the pressure sensitive adhesive includes a (meth)acrylate-based adhesive, a rubber-based adhesive, or a combination thereof.

In certain embodiments, the pressure sensitive adhesive includes a (meth)acrylate-based adhesive (e.g., an isooctyl-acrylate-containing adhesive).

In certain embodiments, at least a portion of the monomers used in making a suitable (meth)acrylate-based adhesive includes alkyl (meth)acrylate monomers having C8-C12 alkyl groups. Examples of such (C8-C12) alkyl (meth)acrylate monomers include 2-ethylhexyl (meth)acrylate, octyl (meth)acrylate, isooctyl (meth)acrylate, nonyl (meth)acrylate, isononyl (meth)acrylate, decyl (meth)acrylate, isodecyl (meth)acrylate, undecyl (meth)acrylate, dodecyl (methacrylate), and the like. Combinations of these alkyl (meth)acrylate monomers can be used if desired.

In certain embodiments, at least a portion of the monomers used in making a suitable (meth)acrylate-based adhesive includes alkyl (meth)acrylate monomers having C1-C7 alkyl groups. Examples of such (C1-C7) alkyl (meth)acrylate monomers include methyl (meth)acrylate, ethyl (meth)acrylate, n-propyl (meth)acrylate, isopropyl (meth)acrylate, n-butyl (meth)acrylate, isobutyl (meth)acrylate, sec-butyl (meth)acrylate, t-butyl (meth)acrylate, pentyl (meth)acrylate, neopentyl (meth)acrylate, hexyl (meth)acrylate, heptyl (meth)acrylate, and the like. In certain embodiments, the monomers include alkyl (meth)acrylate monomers having C4-C6 alkyl groups. Combinations of these alkyl (meth)acrylate monomers can be used if desired.

In certain embodiments, at least a portion of the monomers used in making a suitable (meth)acrylate-based adhesive includes acid-functional monomers. Examples of acid-functional monomers include acrylic acid, methacrylic acid, itaconic acid, itaconic anhydride, crotonic acid, maleic acid, maleic anhydride, 2-(meth)acryloyloxyethyl phthalic acid, 2-(meth)acryloyloxyethyl hexahydrophthalic acid, and the like. Combinations of these acid-functional monomers can be used if desired.

In certain embodiments, the pressure sensitive adhesive includes a rubber-based adhesive including unsaturated olefin-based elastomers such as styrene-butadiene rubbers (SBR).

In certain embodiments, the pressure sensitive adhesive layer has a uniform thickness throughout its length and width. In certain embodiments, the pressure sensitive adhesive layer has a thickness of at least 0.00001 inch (0.00025 mm), or at least 0.001 inch (0.025 mm). In certain embodiments, the pressure sensitive adhesive layer has a thickness of up to 0.01 inch ((0.25 mm), or up to 0.005 inch (0.13 mm).

Additional Optional Layers

The patches of the present disclosure may contain one or more additional layers if desired.

In certain embodiments, the reservoir layer may have an outer surface modified with a slip agent (e.g., an agent such as PVDF or a fluorosilicone to form a slippery surface).

In certain embodiments, a removable siliconized liner may be disposed on the reservoir layer (i.e., a reservoir liner) and/or on the adhesive layer (i.e., an adhesive liner). During use, the adhesive liner is removed from adhesive layer, and the adhesive layer is placed in contact with the wearer's body. Also during use, to allow for release of the insect repellent, the reservoir liner is removed.

The removable liner can be formed of various layers including paper or paper-containing layers or laminates, various thermoplastics such as polyurethane, polyethylene, or polyester films, foil liners, fabric layers coated with, or laminated to, various polymers, etc.

In certain embodiments, the removable liner has a release layer coating disposed on a substrate. The removable liner can be formed of various layers including paper or paper-containing layers or laminates, various thermoplastics such as polyurethane, polyethylene, or polyester films, foil liners, fabric layers coated or laminated to various polymers, etc.

Suitable substrates include, but are not limited to, paper such as poly-coated Kraft paper and super-calendered or glassine Kraft paper; cloth (fabric); nonwoven web; metal or metal alloy including metal foil; polyesters such as poly(alkylene terephthalate) such as poly(ethylene terephthalate), poly(alkylene naphthalate) such as poly(ethylene naphthalate); polycarbonate; polyolefins such as polypropylene, polyethylene, polybutylene, and copolymers thereof; polyamide; cellulosic materials such as cellulose acetate or ethyl cellulose; and combinations thereof.

In certain embodiments, the patches of the present disclosure may contain one or more aesthetic modification layers. Typically, such layers are positioned between the support film and the reservoir layer (i.e., under the reservoir layer), although they may be positioned between the support film and the adhesive layer. Such layer can include decorative indicia (e.g., giving the appearance of a tattoo) if the overlying layer(s) are transparent or colorless. Exemplary layers include nonwoven fabric, such as that available under the tradename SONTARA from DuPont. Patches that include such aesthetic modification layer(s) may further include a second adhesive (e.g., acrylate) to hold such layer(s) in place.

In certain embodiments, the patches of the present disclosure may contain a handling layer. Such handling layer (i.e., carrier layer) can be used to facilitate handling and positioning of the patch to a subject. A similar carrier layer is described in U.S. Pat. No. 6,352,548 (Blach et al.).

Patch Configurations and Methods of Making Patches

Patches of the present disclosure may be fabricated using conventional coating and laminating techniques, including for example, film casting, melt extrusion, etc.

Patches can be of a wide variety of configurations (e.g., size and shape), depending on the use and the wearer (e.g., horse). For example, rounded corners are typically preferred in all exemplary configurations.

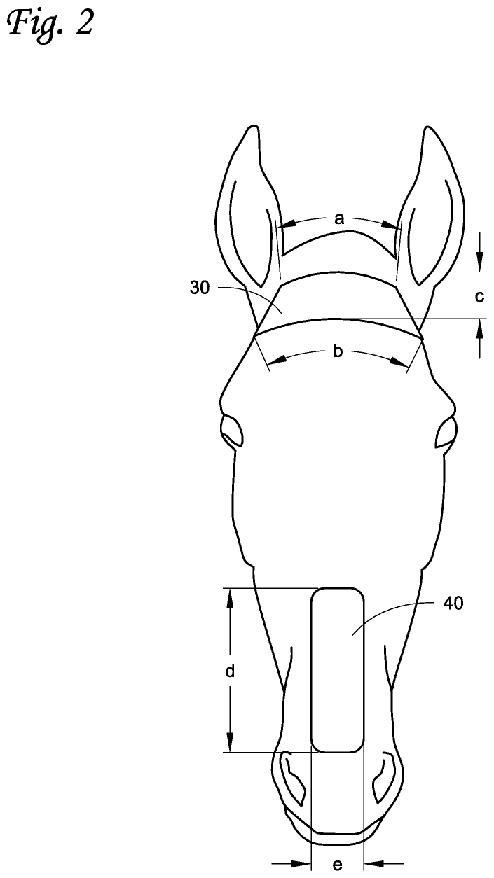

FIGS. 2-7 provide exemplary embodiments of an insect repellent patch configured for use on a horse. It will be appreciated that these configurations are applicable for other quadruped animals. Each of the embodiments includes a support film having a first major surface and a second major surface; a body-contacting pressure sensitive adhesive layer disposed on the first major surface; and a reservoir layer disposed on the second major surface, wherein the reservoir layer comprises an insect repellent incorporated within a polymeric carrier, and wherein the reservoir layer forms a non-contacting surface. The patch sizes and shapes are configured to provide an advantageous plume of the insect repellent over a desired area of the body of a wearer, thereby providing insect repellent to the wearer.

In certain embodiments, the patch is configured of a size and shape for a horse's face. In one embodiment, the patch is a pentagonal configuration, which, for example, is shaped to follow contours of a mid to caudal region of a horse's face. In another embodiment, the patch is a trapezoidal configuration, which, for example, is shaped to follow a poll region of a horse's face. Such configurations are designed to repel insects particularly from the eyes and ears of the animal.

Referring to FIG. 2, patches 30 and 40 are each configured to provide repellent protection to different areas of the horse's face. The dimensions below are not meant to be restrictive but rather, exemplary. Patch 30 is configured to be positioned in the poll region and to repel insects particularly from the eyes and ears of the animal. In the illustrated embodiment for this region, an exemplary trapezoidal configuration is shown having a dimension (length) “a” of 7-20 cm, typically 10-15 cm, for an adult horse, a dimension (length) “b” of 15-25 cm, typically 15-20 cm, for an adult horse, and a dimension (width) “c” of 3-15 cm, typically 7-9 cm, for an adult horse.

Patch 40 is configured to be positioned over the nasal bone region and to repel insects particularly from the eyes and nose of the animal. In this region, a trapezoidal configuration or rectangular configuration is beneficial. In the illustrated embodiment, an exemplary rectangular configuration is shown having a dimension (length) “d” of 5-20 cm, typically 10-15 cm, for an adult horse, and a dimension (width) “e” of 3-12 cm, typically about 6-8 cm, for an adult horse.

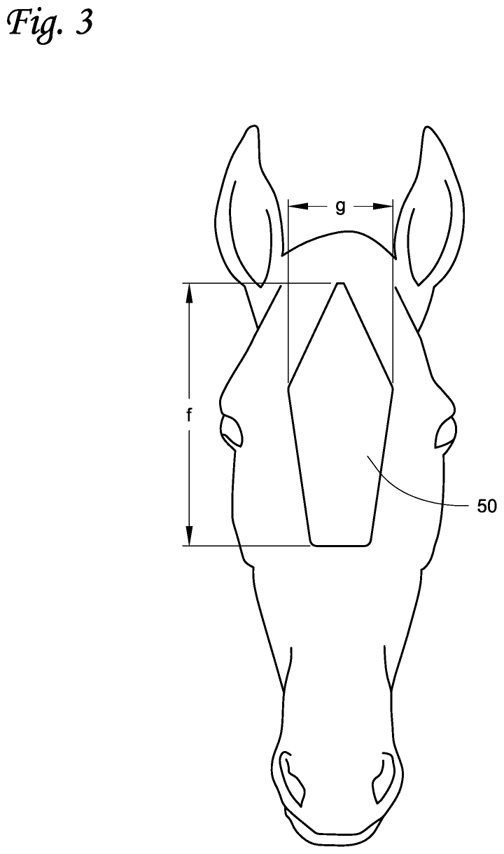

Referring to FIG. 3, patch 50 provides another configuration that follows the contours of the mid to caudal region of the horse's face. In the illustrated embodiment, an exemplary pentagonal configuration is shown having a dimension (length) “f” of 15-25 cm, typically 12-20 cm, for an adult horse, and a dimension (width) “g” of 8-15 cm, typically 10-12 cm, for an adult horse.

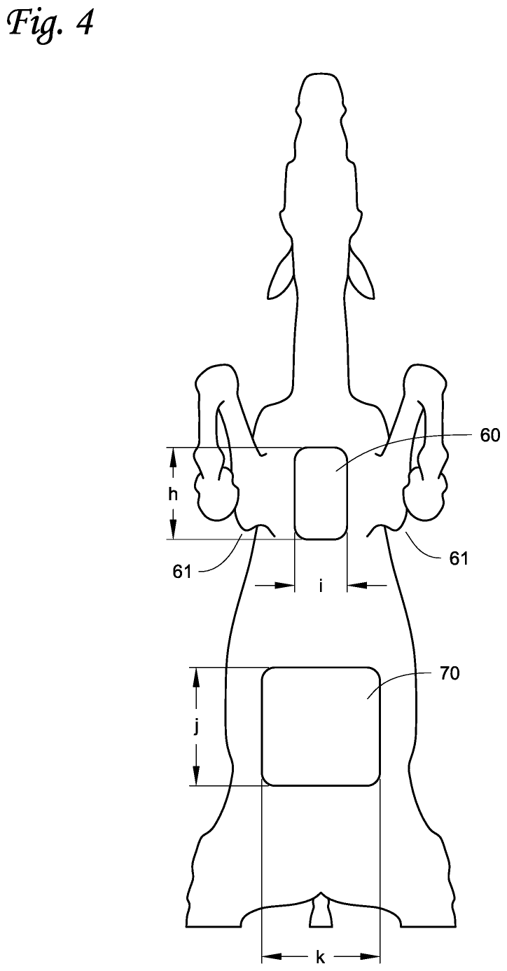

FIG. 4 is a view of the ventral aspect of a horse's body to illustrate relative location and configurations for ventral chest patch 60 and ventral abdominal patch 70. Ventral chest patch 60 can be positioned just cranial of the horse's elbows in the area where the girth strip would be positioned and forward up the chest. Exemplary dimensions include a dimension (length) “h” of ventral chest patch 60 of 14-30 cm, typically 16-20 cm, for an adult horse, and a dimension (width) “i” of 6-25 cm, typically 10-18 cm, for an adult horse.

Ventral abdominal patch 70 can be positioned just cranial of prepuce of male horses (udder on female horses) and extending along the entire ventral surface. In certain embodiments, as ventral abdominal patch 70 and ventral chest patch 60 could be a continuous patch along the entire length of the abdomen (not illustrated). In some embodiments, however, having two separate patches can make application easier, as well as minimize location of the patch beneath girth straps.

In the case of exemplary ventral abdominal patch 70 (FIG. 4), exemplary dimensions include a dimension (length) “j” of 25-50 cm, typically 25-35 cm, for an adult horse, and a dimension (width) “k” of 25-50 cm, typically 25-35 cm, for an adult horse. While not illustrated, the width dimension of ventral abdominal patch 70 can extend laterally up the side walls of the horse.

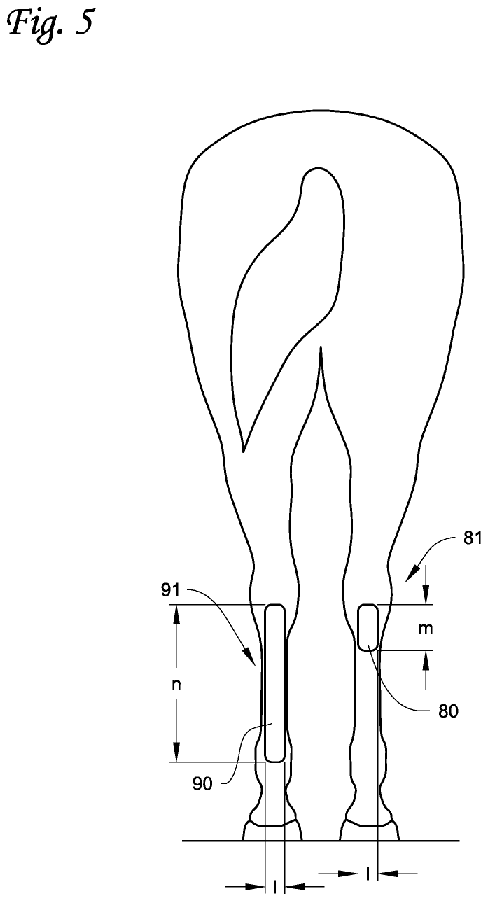

FIG. 5 is a rear view of a horse showing two different sizes and configurations for tarsal repellent patch 80 for applying over the tarsal joint region 81 of the rear leg, and plantar repellent patch 90 for applying over the entire plantar surface 91 of the rear leg. Exemplary dimension (width) “1” for patches 80 and 90 are 1-6 cm, typically 2-3 cm, for an adult horse. Exemplary dimension (length) “m” for plantar repellent patch 80 is 5-15 cm, typically 4-6 cm, for an adult horse. Exemplary dimension (length) “n” for plantar repellent patch 90 is 10-40 cm, typically 20-30 cm, for an adult horse.

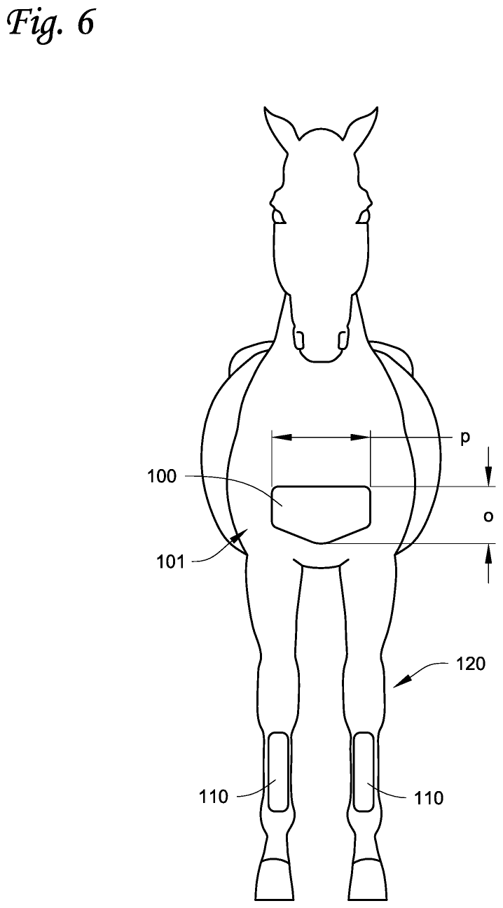

FIG. 6 is a front view of a chest repellent patch 100 applied to the anterior chest region 101 of an adult horse. In the illustrated embodiment, an exemplary configuration is shown having a dimension “o” of 12-25 cm, typically 15-20 cm, for an adult horse, and a dimension “p” of 6-25 cm, typically 10-16 cm, for an adult horse.

Also shown in FIG. 6, cannon bone repellent patches 110 are shown. These are similar in width to plantar repellent patch 90 (FIG. 5) and the length is often slightly shorter than the length of the plantar repellent patch 90, consistent with typical relative dimensions of the front and rear legs of a horse. Patches configured for the carpal region 120 can have a similar length to tarsal repellent patch 80 (FIG. 5). Although not illustrated, carpal patches can have a width dimension broader than tarsal repellent patch 80. It will be appreciated that patches configured for horses on the front and back surfaces of the front and rear legs above the carpal and tarsal joints, respectively, are within the scope of the disclosure.

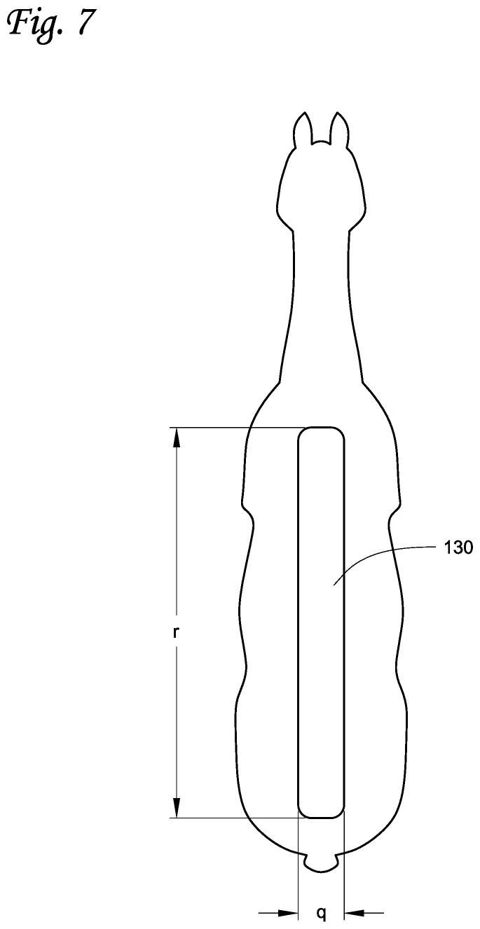

FIG. 7 is a top view of a horse illustrating a top-line repellent patch 130 applied to the back of an adult horse. In the illustrated embodiment, an exemplary rectangular configuration is shown having a dimension (width) “q” of 12-25 cm, typically 15-20 cm, for an adult horse, and a dimension (length) “r” of 40-100 cm, typically 40-60 cm, for an adult horse.

In some embodiments, repellent patches disclosed herein are configured to provide a plume of repellent vapor extending a distance of 0.1-100 cm, or 1-80 cm, or 1-50 cm, or 2-30 cm, from the surface of the patch.

Tubular Repellent Articles

An insect repellent tubular article (i.e., tubular repellent article) includes: a tubular structure having a first major surface and a second major surface, wherein the tubular structure comprises an elastic or shape memory material; wherein the first major surface provides a reservoir comprising an insect repellent, and the second major surface provides a body-contacting surface. In certain embodiments, the body-contacting surface of the tubular structure is coated with a barrier.

Typically, a tubular article extends the length of an animal's leg from the hoof or coronet band at the distal aspect to above or just below the elbow (front leg) or stifle (rear leg) proximally.

The “tubular repellent article” or “repellent tubular article” can be configured to conform to the shape of the animal's leg using an elastic or shape memory material such as polyether-polyurea co-polymers (spandex, Lycra®, or elastane). In certain embodiments, the elastic or shape memory material (preferably spandex) can be woven into known synthetic, semi-synthetic, or organic fibers. The ratio of the elastic or shape memory material (preferably spandex) used when woven with other fabrics preferably maintains 4 way stretch and provides for the tubular repellent article to remain fully extended along the length of the animal's leg when the animal moves, at least, at a walk. For example, in some embodiments the tubular repellent can be 10%-90%, 15-20%, 25-30%, or 50% or more elastic or shape memory material (preferably, spandex). One hundred percent of the elastic or shape memory material (preferably, spandex) may be used, however, weaving with other fabrics may provide additional benefits. It is desirable that if woven with other fibers, sufficient spandex is present to maintain properties of breathability, moisture wicking, and stretch ability. Nylon, polyester, cotton, and wool are non-limiting examples of fibers to be woven with the spandex.

In tubular repellent articles, the insect repellents disclosed herein with or without synergists may be used. The tubular repellent article includes a first major surface and a second major surface. The first major surface provides a reservoir of insect repellent that is adsorbed to, absorbed to, impregnated or otherwise attached to the first major surface of the elastic or shape memory material (preferably spandex). The insect repellent can be impregnated using known systems such as that provided by INSECT SHIELD technology. The first major surface may provide insect repellent quantities to provide for multiple washings. The first major surface may also provide for “reloading” of the insect repellent onto the first major surface using, for example, insect repellent sprays.

In certain embodiments, the second major surface contacts, for example, an animal's leg and may be coated with a barrier to reduce the likelihood of impregnated insect repellent being released from the second major surface. Suitable materials may be selected from the Support Materials provided herein above and applied using known methods including solubilization in a solvent and spray coating or heat lamination. In embodiments, the second major surface can comprise a fabric such as a non-woven material that is in contact with the body of the animal.

In certain embodiments, a tubular repellent article may be configured to the shape of a horse's head. It can have cut-outs or openings to avoid covering the horse's eyes, mouth and/or nose. The tubular repellent article is configured to minimize the ability of insects to enter the tubular structure between the bones of the horse's mandible and other areas around the ends of the tubular repellent include, for example increased elasticity or an adhesive. Suitable adhesives for adhering to the animal's body are disclosed herein.

In certain embodiments, a tubular repellent article may include “ribs” to provide spacing between the second major surface and the horse's body.

FIG. 8 is a lateral view of a horse's body showing a tubular repellent article according to the disclosure. Front leg 200 is covered by tubular repellent article 201. The tubular repellent article 201 is configured to conform to the contours of the animal's front leg such that the tube does not significantly slide down the leg from its full coverage position when the animal walks. Tubular repellent article 201 can have an increased amount of elasticity at the top region 202 and/or bottom region 203 to prevent insects from entering the tubular structure and help reduce the likelihood of the tubular repellent article sliding distally once in position. Alternatively, or in addition, top region 202 and/or bottom region 203 can include a pressure sensitive adhesive as disclose herein for the patches to further reduce the likelihood of distal slippage of the tubular repellent article when in use.

Still referring to FIG. 8, a rear leg 210 is covered by tubular repellent article 211 similar to that disclosed for the front leg 200. The tubular repellent article 211 is configured to conform to the contours of the horse's rear leg such that the tube does not slide distally when the animal walks. Tubular repellent article 211 can have an increased amount of elasticity at the top region 212 and/or bottom region 213 to prevent insects from entering the tubular structure and reduce the likelihood of the tubular repellent article sliding distally once in position. Alternatively, or in addition, top region 212 and/or bottom region 213 can include a pressure sensitive adhesive as disclose herein for the patches to further reduce the likelihood of distal slippage of the tubular repellent article when in use.

In a similar manner a tubular repellent article 300 configured to snugly fit on the horse's head 310 is shown. Cut out 302 for the horse's eye and 303 for the horse's mouth can be provided using known methods.

FIG. 9 is a cross-section view of tubular repellent article 201 at line 9-9. As illustrated, ribs 204 are positioned around the circumference of tubular repellent article 201 that provide spacing between the second major surface 205 and the animals fur when in use. The ribs can be located along some or all of the length of the tubular repellent. The ribs can be formed by extrusion or adhesion. Ribs can be my from, for example, polyurethane. The ribs increase the distance between the insect repellent impregnated shape memory material and the animal's body. Not only does this reduce the likelihood of contact of insect repellent with animal's skin but it also provides a gap to increase the distance between the second major surface and the animal's skin if an insect does land on the tubular repellent article and try to bite the animal.

Insects

Many insects are well known pests to humans and animals. A few examples of pests that bother horses are provided to serve as non-limiting examples of insects that the articles of the present disclosure effectively repel.

Face flies (Musca autumnalis) are a non-biting fly that feed on facial secretions of animals. Female adult face flies are oftent found around horses' eyes, mouth, and muzzle, causing immense annoyance.

Stable flies (Stomoxys calcitrans) are blood-feeding flies that are often found feeding on the legs of horses. Stable flies are the major cause of horses kicking and stomping, which happens when the flying is feeding on the horse. They have piercing, sucking-type mouthparts that inflict a painful bite and are responsible for spreading certain diseases including equine infectious anemia.

Horse and deer flies are in the Tabanidae family. Horse and deer flies are larger than most of the other flies discussed here. Females have knifelike mouthparts that can inflict an extremely painful bite and leave behind bloody feeding sites.

Horn flies (Haematobia irritans) are small flies that are primarily a pest of pastured cattle, but are also found around horses, sheep, and goats. Horn flies show up on the backs, shoulders and sides of the animal. Both male and female horn flies have piercing mouthparts and feed on blood up to 40 times per day.

Other exemplary pests include mosquitos, biting midges, gnats and others common to bothering humans and animals.

### EXEMPLARY EMBODIMENTS

Embodiment 1 is an insect repellent patch comprising: a support film having a first major surface and a second major surface; a body-contacting pressure sensitive adhesive layer disposed on the first major surface; and a reservoir layer disposed on the second major surface, wherein the reservoir layer comprises an insect repellent incorporated within a polymeric carrier; wherein the reservoir layer forms a non-contacting surface.

Embodiment 2 is an insect repellent patch comprising: an occlusive support film having a first major surface and a second major surface; a body-contacting pressure sensitive adhesive layer disposed on the first major surface; and a reservoir layer disposed on the second major surface, wherein the reservoir layer comprises an insect repellent incorporated within a nonporous diffusion controlling polymeric carrier; wherein the reservoir layer forms a non-contacting surface.

Embodiment 3 is the patch of embodiment 1 or 2, wherein the polymeric carrier, insect repellent, and support film are selected to pass the Adhesion Test.

Embodiment 4 is the patch of any one of the previous embodiments, wherein the polymeric carrier, insect repellent, and support film are selected to pass the Tack Test.

Embodiment 5 is the patch of any one of the previous embodiments, which is effective for repelling insects for a period of at least 1 day, at least 2 days, at least 7 days, at least 14 days, or at least 1 month.

Embodiment 6 is the patch of any one of the previous embodiments, which is effective for repelling insects for a period of up to 3 months, up to 21 days, up to 14 days, or up to 7 days.

Embodiment 7 is the patch of any one of the previous embodiments, wherein the support film is sufficiently flexible to conform to a wearer's body contours.

Embodiment 8 is the patch of embodiment 7, wherein the support film comprises an organic polymer (e.g., thermoplastic resin or thermoset resin), a metal-containing material (e.g., metal such as A1 or other thin metal film, metal alloy, vapor-deposited metal layer, or shape memory metal), or a combination thereof.

Embodiment 9 is the patch of embodiment 8, wherein the support film is formed from a thermoplastic resin.

Embodiment 10 is the patch of embodiment 9, wherein the thermoplastic resin is selected from poly(meth)acrylates (such as the polymethylmethacrylate, poly-n-butyl acrylate, poly(ethylene-co-acrylic acid), poly(ethylene-co-methacrylate), etc.), fluoropolymers (such as polytetrafluoroethylene (PTFE) (e.g., that available under the tradename TEFLON), poly(ethylene-co-tetrafluoroethylene) (ETFE) copolymers, (tetrafluoroethylene-co-propylene) (FEP) copolymers, polyvinylidine fluoride (PVDF) polymers, etc.), perfluoroalkoxy alkanes (PFA) polymers, polyamides (such as nylon-6, nylon-6,6, etc.), polycarbonates, polyesters (such as poly(ethylene-co-terephthalate), poly(ethylene-co-1,4-naphthalene dicarboxylate), polysulfones, polyketones, polyurethanes, polyimide materials, poly(acrylonitrile-co-butadiene-co-styrene) polymers, poly(butylene-co-terephthalate), polyethylene materials (such as low density polyethylene, linear low density polyethylene, high density polyethylene, high molecular weight high density polyethylene, etc.), polypropylene (such as biaxially oriented polypropylene), polystyrene (such as biaxially oriented polystyrene), vinyl resins (such as polyvinyl chloride, (vinyl chloride-co-vinyl acetate) copolymers, polyvinylidene chloride, polyvinyl alcohol, (vinyl chloride-co-vinylidene dichloride) copolymers), polyphenylene sulfides, polyphenylene oxides, liquid crystal polyesters, polyether ketones, polyvinylbutyral, and combinations thereof.

Embodiment 11 is the patch of embodiment 10, wherein the thermoplastic resin is selected from poly(meth)acrylates (such as the polymethylmethacrylate, poly-n-butyl acrylate, poly(ethylene-co-acrylic acid), poly(ethylene-co-methacrylate), etc.), fluoropolymers (such as polytetrafluoroethylene (PTFE) (e.g., that available under the tradename TEFLON), poly(ethylene-co-tetrafluoroethylene) (ETFE) copolymers, (tetrafluoroethylene-co-propylene) (FEP) copolymers, polyvinylidine fluoride (PVDF) polymers, etc.), perfluoroalkoxy alkanes (PFA) polymers, polyamides (such as nylon-6, nylon-6,6, etc.), polycarbonates, polyesters (such as poly(ethylene-co-terephthalate), poly(ethylene-co-1,4-naphthalene dicarboxylate), polysulfones, polyketones, polyurethanes, polyimide materials, and combinations thereof.

Embodiment 12 is the patch of embodiment 11, wherein the thermoplastic resin is a polyester (such as polyethylene terephthalate).

Embodiment 13 is the patch of any one of the previous embodiments, wherein the support film has a uniform thickness throughout its length and width.

Embodiment 14 is the patch of any one of the previous embodiments, wherein the support film has a thickness of at least 0.008 inch (0.20 mm)

Embodiment 15 is the patch of any one of the previous embodiments, wherein the support film has a thickness of up to 0.020 inch (0.52 mm).

Embodiment 16 is the patch of any one of the previous embodiments, wherein either or both of the first major surface and second major surface of the support film is a treated surface.

Embodiment 17 is the patch of embodiment 16, wherein the treated surface is corona treated, plasma treated, or chemically treated to enhance adhesion of the layer(s) disposed thereon (e.g., the adhesive layer and the reservoir layer).

Embodiment 18 is the patch of any one of the previous embodiments, wherein the pressure sensitive adhesive layer is directly disposed on the first major surface of the support film.

Embodiment 19 is the patch of any one of the previous embodiments, wherein the reservoir layer is directly disposed directly on the second major surface of the support film.

Embodiment 20 is the patch of any one of the previous embodiments, wherein the pressure sensitive adhesive is selected to adhere to a horse body (particularly horse hair).

Embodiment 21 is the patch of any one of the previous embodiments, wherein the pressure sensitive adhesive comprises a (meth)acrylate-based adhesive, a rubber-based adhesive, or a combination thereof.

Embodiment 22 is the patch of embodiment 21, wherein the pressure sensitive adhesive comprises a (meth)acrylate-based adhesive.

Embodiment 23 is the patch of any one of the previous embodiments, wherein the pressure sensitive adhesive layer has a uniform thickness throughout its length and width.

Embodiment 24 is the patch of any one of the previous embodiments, wherein the pressure sensitive adhesive layer has a thickness of at least 0.00001 inch (0.00025 mm), or at least 0.001 inch (0.025 mm).

Embodiment 25 is the patch of any one of the previous embodiments, wherein the pressure sensitive adhesive layer has a thickness of up to 0.01 inch (0.25 mm), or up to 0.005 inch (0.13 mm).

Embodiment 26 is the patch of any one of the previous embodiments, wherein the polymeric carrier of the reservoir layer is selected to adhere to the support layer but not adhere to environmental contaminants (e.g., sand, dust, dirt, debris, and hair).

Embodiment 27 is the patch of any one of the previous embodiments, wherein the polymeric carrier of the reservoir layer is selected to solubilize the insect repellent and be permeable to the insect repellent.

Embodiment 28 is the patch of any one of the previous embodiments, wherein the polymeric carrier of the reservoir layer comprises a rubber-based polymer, an unsaturated polyolefin (such as polyisobutene, polybutadiene, cis- and trans-polyisoprene, and chloroprene elastomers), a copolymer of unsaturated polyolefins (such as styrene-butadiene copolymer, styrene-isoprene copolymer, and acrylonitrile-butadiene-styrene copolymers), a vinyl polymer (such as polyvinyl chloride), a (meth)acrylate (such as poly-n-butyl acrylate), or a combination thereof.

Embodiment 29 is the patch embodiment 28, wherein the polymeric carrier of the reservoir layer comprises a rubber-based polymer, an unsaturated polyolefin, a copolymer of unsaturated polyolefins, or a combination thereof.

Embodiment 30 is the patch of embodiment 29, wherein the polymeric carrier comprises polyisobutene, polybutadiene, polystyrene-isoprene copolymer, polystyrene-butadiene (SBR) copolymer, polychloroprene (neoprene), isoprene (i.e., 2-methyl-1,3-butadiene), or a combination thereof.

Embodiment 31 is the patch of embodiment 30, wherein the polymeric carrier comprises a polystyrene-butadiene (SBR) copolymer.

Embodiment 32 is the patch of any one of the previous embodiments, wherein the reservoir layer has an outer surface modified with a slip agent (e.g., an agent such as PVDF or a fluorosilicone to form a slippery surface).

Embodiment 33 is the patch of any one of the previous embodiments, wherein the polymeric carrier of the reservoir layer is crosslinked.

Embodiment 34 is the patch of any one of the previous embodiments, wherein the reservoir layer is coextensive with the support film (i.e., the reservoir layer is disposed on the entirety of the support film).

Embodiment 35 is the patch of any one of the previous embodiments, wherein the reservoir layer does not include a fibrous material.

Embodiment 36 is the patch of any one of the previous embodiments, wherein the reservoir layer does not include a foam pad.

Embodiment 37 is the patch of any one of the previous embodiments, wherein the reservoir layer has a uniform thickness throughout its length and width.

Embodiment 38 is the patch of any one of the previous embodiments, wherein the reservoir layer has a thickness of at 0.005 inch (0.013 mm).

Embodiment 39 is the patch of any one of the previous embodiments, wherein the reservoir layer has a thickness of up to 0.025 inch (0.64 mm).

Embodiment 40 is the patch of any one of the previous embodiments, wherein the insect repellent has a vapor pressure sufficient to allow the insect repellent to diffuse out of the reservoir layer.

Embodiment 41 is the patch of any one of the previous embodiments, wherein the insect repellent releases from the reservoir layer over a period of at least 1 day, at least 2 days, at least 7 days, at least 14 days, or at least 1 month.

Embodiment 42 is the patch of any one of the previous embodiments, wherein the insect repellent releases from the reservoir layer over a period of up to 3 months, up to 21 days, up to 14 days, or up to 7 days.

Embodiment 43 is the patch of any one of the previous embodiments, wherein the insect repellent comprises a combination of compounds that effectively repel insects.

Embodiment 44 is the patch of any one of the previous embodiments, wherein the insect repellent is present in the polymeric carrier of the reservoir layer in an amount of at least 1 wt-%, or at least 5 wt-%, or at least 10 wt-%, based on the total weight of the reservoir.

Embodiment 45 is the patch of any one of the previous embodiments, wherein the insect repellent is present in the polymeric carrier of the reservoir layer in an amount of up to 90 wt-%, or up to 85 wt-%, or up to 80 wt-%, or up to 75 wt-%, or up to 70 wt-%, or up to 65 wt-%, based on the total weight of the reservoir.

Embodiment 46 is the patch of any one of the previous embodiments, wherein the insect repellent comprises a naturally occurring compound, a synthetic compound, or a combination thereof.

Embodiment 47 is the patch of any one of the previous embodiments, wherein the insect repellent comprises a naturally occurring pyrethrum extract, a synthetic pyrethroid, a naturally occurring oil, a terpene, a terpenoid, an alkanoic acid, an organophosphate, a toluamide, or a combination thereof.

Embodiment 48 is the patch of embodiment 47, wherein the insect repellent comprises a naturally occurring pyrethrum extract, a synthetic pyrethroid, or a combination thereof.

Embodiment 49 is the patch of embodiment 48, wherein the insect repellent comprises permethrin, cypermethrin, metofluthrin, profluthrin, transfluthrin, or a combination thereof. Embodiment 50 is the patch of embodiment 49, wherein the insect repellent comprises a naturally occurring oil.

Embodiment 51 is the patch of embodiment 50, wherein the naturally occurring oil comprises lemon eucalyptus oil, clove oil, cottonseed oil, neem oil, lemongrass oil, citronella oil, cedarwood oil, lavender oil, rosemary oil, coconut oil, fir needle oil, litsea cubeba oil, pyganic (chrysanthemum) oil, or a combination thereof.

Embodiment 52 is the patch of embodiment 47, wherein the insect repellent comprises a terpene (e.g., pulegone), a terpenoid (e.g., geraniol), an alkanoic acid (e.g., a C8-C10-alkanoic acid), an organophosphate (e.g., 2,2-dichlorovinyl dimethyl phosphate), a toluamide (e.g., N,N-diethyl-meta-toluamide (i.e., DEET)), or a combination thereof.

Embodiment 53 is the patch of any one of the previous embodiments, wherein the reservoir layer further comprises an additive selected from a tackifier, plasticizer, UV absorber, antioxidant, sunscreen, synergist, stabilizer, or a combination thereof.

Embodiment 54 is the patch of embodiment 53, wherein the reservoir layer further comprises a synergist.

Embodiment 55 is the patch of embodiment 54, wherein the synergist is selected from piperonyl butoxide, di-n-propyl isocynchomeronate, buoxypolypropylene glycol, bicycloheptene dicaroxamide, and a combination thereof.

Embodiment 56 is the patch of embodiment 53, wherein the reservoir layer further comprises a tackifier.

Embodiment 57 is the patch of embodiment 56, wherein the tackifier is selected from a rosin or derivative thereof, a terpene or modified terpene, an aliphatic resin (e.g., C5-aliphatic resin), a cycloaliphatic resin (e.g., C6-cycloaliphatic resin), an aromatic resin (e.g., C9-aromatic resin), a hydrogenated hydrocarbon resin, a novolac resin, and a combination thereof (e.g., C5/C9 aliphatic/aromatic resin, terpene-phenol (TPR) resin).

Embodiment 58 is the patch of embodiment 53, wherein the reservoir layer further comprises a plasticizer.

Embodiment 59 is the patch of embodiment 58, wherein the plasticizer is selected from a phthalate, a trimellitate, an adipate, and a combination thereof.

Embodiment 60 is the patch of embodiment 53, wherein the reservoir layer further comprises a stabilizer.

Embodiment 61 is the patch of embodiment 60, wherein the stabilizer is selected from a light stabilizer (e.g., oxybenzone), antioxidant (e.g., hindered amine and phenol), and a combination thereof.

Embodiment 62 is the patch of any one of the previous embodiments, further comprising a removable siliconized liner is disposed on the reservoir layer.

Embodiment 63 is the patch of any one of the previous embodiments, further comprising a removable siliconized liner is disposed on the adhesive layer.

Embodiment 64 is the patch of any one of the previous embodiments, further comprising an aesthetic modification layer under the reservoir layer.

Embodiment 65 is the patch of any one of the previous embodiments, further comprising a handling layer.

Embodiment 66 is the patch of any one of the previous embodiments, which is of a size and shape for a horse's face (to provide the insect repellent to the horse's face, particularly eyes and ears of the horse).

Embodiment 67 is the patch of embodiment 66, which is a pentagonal configuration.

Embodiment 68 is the patch of embodiment 67, which is shaped to follow contours of a mid to caudal region of a horse's face.

Embodiment 69 is the patch of embodiment 66, which is a trapezoidal configuration.

Embodiment 70 is the patch of embodiment 69, which is shaped to follow a poll region of a horse's face.

Embodiment 71 is the patch of any one of the previous embodiments, which is configured to provide a plume of insect repellent vapor extending a distance of 0.1-100 cm, or 1-80 cm, or 1-50 cm, or 2-30 cm, from the surface of the patch.

Embodiment 72 is an insect repellent tubular article that includes: a tubular structure having a first major surface and a second major surface, wherein the tubular structure comprises an elastic or shape memory material; wherein the first major surface provides a reservoir comprising an insect repellent, and the second major surface provides a body-contacting surface.

Embodiment 73 is the insect repellent tubular article of embodiment 72 wherein the body-contacting surface of the tubular structure is coated with a barrier.

Embodiment 74 is the insect repellent tubular article of embodiment 72 or 73 that extends the length of an animal's leg from the hoof or coronet band at the distal aspect to above or just below the elbow (front leg) or stifle (rear leg) proximally.

Embodiment 75 is the insect repellent tubular article of any one of embodiments 72 through 74 further comprising “ribs” to provide spacing between the second major surface (i.e., the body-contacting surface and the wearer's body (e.g., horse's body).

### EXAMPLES

These examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding.

Materials

| Material | Product Information |
|---|---|
| PET Tape | Shipping packaging tpe heavy duty, acrylate-based |
| | adhesisve, (1.9 inch (48 mm) × 0.0031 inch (0.079 |
| | mm)), 3M Company (St. Paul, MN) |
| Polymeric | CAMIE 390B, polystyrene-butadiene (SBR) copolymer, |
| Carrier 1 | (17% solids), Ellsworth Adhesives (Germantown, WI) |
| Polymeric | Ethylene vinyl acetate (EVA) hot melt adhesive, Bostik |
| Carrier 2 | Adhesives (Wauwatosa, WI) |
| (Comparative) | |
| Polymeric | SC1329 natural rubber-based, isoprene, adhesive, H. B. |
| Carrier 3 | Fuller (St. Paul, MN) |
| Pyrethrum | >50% sum of pyrethrins product number 82670, Sigma |
| extract | (St. Louis, MO) |
| Piperonyl | 90% product number 291102, Sigma-Aldrich |
| Butoxide | |
| Geraniol | 98% product number 163333, Sigma-Aldrich |
| DEET | Sigma-Aldrich |

Preparation of Patches

Solutions for the reservoir layer were prepared at an equivalent 15% solids using toluene to adjust the concentration. Samples of 0.75 inch (19 mm) diameter were coated in circular molds onto the PET tape to a dry thickness of approximately 0.020 inch (0.51 mm). The insect repellent was formulated at 2% within the dry carrier.

Subsequent to coating, the samples were first allowed to air dry at room temperature for approximately 1 hour, then placed in a heated air convection oven at approximately 100F for several hours. Upon removal, samples sat overnight before evaluation.

Tack Test

As a measure of tack, the surface of the coated formulation was probed with the flat end of a wooden dowel The wooden dowel was applied under hand pressure to the surface of the reservoir layer for 1 second long in contact. A positive (+) result for tack indicates there was no transfer of the carrier polymer of the reservoir layer to the probe (adhesive failure).

Adhesion Test

A sharpened to a point like a flat wooden dowel (approximately 3 mm diameter) was used to separate the reservoir layer/support film coating interface. The sharpened end of the wooden dowel was applied under hand pressure at the interface in an attempt to separate the reservoir layer from the support film. A positive (+) result for adhesion indicates the carrier cohesively failed with transfer of polymer carrier material of the reservoir layer to the support film.

Test Results

| | Camie SBR | Bostik EVA | Fuller SC1329 |
|---|---|---|---|
| Pyrethrum extract | adhesion/tack | adhesion/tack | adhesion/tack |
| | + + | − + | − − |
| PBO | + + | − + | − − |
| Geraniol | + + | − + | − − |
| Deet | + + | − + | − − |

The complete disclosures of the patents, patent documents, and publications cited herein are incorporated by reference in their entirety as if each were individually incorporated. To the extent that there is any conflict or discrepancy between this specification as written and the disclosure in any document that is incorporated by reference herein, this specification as written will control. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. An insect repellent patch comprising:
an occlusive support film having a first major surface and a second major surface;
a body-contacting pressure sensitive adhesive layer disposed on the first major surface; and
a reservoir layer disposed on the second major surface, wherein the reservoir layer comprises an insect repellent incorporated within a nonporous diffusion controlling polymeric carrier;
wherein the polymeric carrier comprises a rubber-based polymer selected from polyisobutene, polybutadiene, polystyrene-isoprene copolymer, polystyrene-butadiene (SBR) copolymer, polychloroprene (neoprene), isoprene (i.e., 2-methyl-1,3-butadiene), or a combination on thereof; and
wherein the reservoir layer forms a non-contacting surface.

2. The patch of claim 1, wherein the polymeric carrier, insect repellent, and support film are selected to pass the Adhesion Test.

3. The patch of claim 1, wherein the polymeric carrier, insect repellent, and support film are selected to pass the Tack Test.

4. The patch of claim 1, which is effective for repelling insects for a period of 1 day to 3 months.

5. The patch of claim 1, wherein the support film comprises an organic polymer, a metal-containing material, or a combination thereof.

6. The patch of claim 5, wherein the support film is formed from a thermoplastic resin.

7. The patch of claim 6, wherein the thermoplastic resin is polyethylene terephthalate.

8. The patch of claim 1, wherein the pressure sensitive adhesive is selected to adhere to a horse body.

9. The patch claim 8, wherein the polymeric carrier of the reservoir layer comprises a rubber-based polymer.

10. The patch of claim 1, wherein the insect repellent is present in the polymeric carrier of the reservoir layer in an amount of 1 wt-% to 90 wt-%, based on the total weight of the reservoir.

11. The patch of claim 1, wherein the insect repellent comprises a naturally occurring pyrethrum extract, a synthetic, pyrethroid, a naturally occurring oil, a terpene, a terpenoid, an alkanoic acid, an organophosphate, a toluamide, a naturally occurring oil, a terpene, a terpenoid, alkanoic acid, an organophosphate, a toluamide, or a combination thereof.

12. The patch of claim 1, which is of a size and shape configured for a horse's face.

13. The patch of claim 1, which is configured to provide a plume of insect repellent vapor extending a distance of 0.1-100 cm from the non-contacting surface of the patch.

14. An insect repellent patch comprising:
a support film having a first major surface and a second major surface;
a body-contacting pressure sensitive adhesive layer disposed on the first major surface; and
a reservoir layer disposed on the second major surface, wherein the reservoir layer comprises an insect repellent incorporated within a polymeric carrier; wherein the reservoir layer forms a non-contacting surface;
wherein the patch is a pentagonal or trapezoidal configuration of a size for a horses's face.

15. The patch of claim 14, which is configured to provide a plume of insect repellent vapor extending a distance of 0.1-100 cm from the non-contacting surface of the patch.

16. An insect repellent tubular article comprising:
a tubular structure having a first major surface and a second major surface, wherein the tubular structure comprises an elastic or shape memory material;
wherein the first major surface provides a reservoir comprising an insect repellent, and the second major surface provides a body-contacting surface.

17. The insect repellent tubular article of claim 16 wherein the body-contacting surface of the tubular structure is coated with a barrier.
